(19) United States
(12) Patent Application Publication
(10) Pub. No.: US 2015/0142056 A1
(43) Pub. Date: May 21, 2015

(54) Flexible Facet Screw Apparatus

(71) Applicant: Jerry Hart, Oceanside, CA (US)

(72) Inventor: Jerry Hart, Oceanside, CA (US)

(21) Appl. No.: 14/080,897

(22) Filed: Nov. 15, 2013

(51) Int. Cl. A61B 17/70

(52) U.S. Cl. CPC A61B 17/7064
USPC 606/247; 606/279

(57) ABSTRACT

A novel facet screw that addresses the need to elevate facet related pain caused by the facet faces being near bone on bone and or misaligned by providing an improved facet screw apparatus, and methods for the delivering that screw, and securing the vertebral facets while allowing the center of the screw to become flexible, thereby allowing the facets to move more normal. The present invention comprises a proximal mesh engagement mechanism, an adhesion ring, a distal mesh engagement mechanism, a drive socket bonding core, a flexible mesh, a stabilizing plug and a flexible separation donut. A specially designed drill mechanism is used to pre-drill the holes in the facets for installing the present invention novel facet screw. A solid tightening wrench is utilized clinically to initially install the novel facet screw into two corresponding spinal facets.

### FIELD OF THE INVENTION

This present invention relates to devices for spinal surgical procedures. More specifically, the present invention relates to devices for augmentation and restoration of vertebral facet joints affected by degeneration and the surgical method of implanting these devices in the spine.

### BACKGROUND OF THE INVENTION

Inter-vertebral disc disease is a major worldwide health, problem. In the United States alone almost 600,000 to 700,000 spine procedures are performed each year and the total cost of treatment of back pain exceeds $25 billion. Age related changes in the disc include diminished water content in the nucleus and increased collagen content by the 4^{th} decade of life. Loss of water binding by the nucleus results in more compressive loading of the annulus. This renders the annulus more susceptible to delamination and damage. Damage to the annulus, in turn, accelerates disc degeneration and degeneration of surrounding tissues such as the facet joints.

Various disorders of the spine can lead to severe pain and loss of mobility. According to some studies, back and spinal impairments are the leading causes of lost work productivity in the United States. Pain as a result of some type of spinal impairment may have its source in a wide variety of pathologies or clinical conditions. In many instances, damage to the spine as a result of advancing age, disease and injury is treated by fixation or stabilization of vertebrae. Conventional methods of spinal fixation utilize spinal fusion, a procedure in which bone growth is encouraged to bridge the disc space to fuse together adjacent vertebrae and as a result, stabilize the spinal motion segment. Spinal fusion involves at least partial removal of a damaged intervertebral disc and the introduction of bone graft material that is typically contained in an interbody spacer which is implanted into the intervertebral disc space. Kidney bean-shaped, curved or other shaped interbody spacers or cages made of titanium or polyether ether ketone (PEEK) are employed to provide decompression and house the bone graft material. The bone graft material is usually supplemented with bone morphogenic protein, demineralized bone matrix in the form of paste or cement, stem cells or other oseoinductive biological agents which are known to enhance fusion.

One such source for pain is related to degeneration of the facets of the spine or facet arthritis.

The two most common spinal surgical procedures performed are discectomy, spinal section fusion, and rod and screw bracing of the spine sections. These procedures usually only address the symptom of lower back pain. These procedures can worsen the overall condition of the affected disc and the adjacent discs, and the related facet joints.

There are many different types of spinal fusion procedures. One is anterior lumbar interbody fusion or “ALIF”. In ALIF, the patient is placed on their back and the section of the spine to be fused is approached through the front of the patient through incisions in the abdomen. The abdominal organs are moved aside, the damaged disc space is exposed and bone graft material encaged in an interbody spacer is implanted. Another spinal fusion technique is posterior lumbar interbody fusion (“PLIF”) in which the spine is approached and bone graft material is implanted through the back of a patient lying prone on a surgical table. A type of PLIF is transforaminal lumbar interbody fusion or “TLIF”. TLIF is another widely used method of spinal fusion for the treatment of a variety of lumbar spinal disorders when avoidance of complex anterior approaches and diminished posterior trauma is desired. In TLIF, the spine is approached through the backside of the patient as in PLIF; however, access to the interbody space is gained through the foramina. Another surgical spinal fusion technique is extreme lateral interbody fusion, also known as XLIF, which is performed through the patient's side, avoiding the major muscles of the back. Other surgical spinal fusion techniques include direct lumbar interbody fusion (“DLIF”).

While many technological advances have focused on the spinal disc and artificial replacement or repair of the disc, little advancement in facet restoration has been made. Facet joint and disc degeneration frequently occur together. Therefore, there is a need to address the clinical concerns related to degenerative facet joints and the pain emanating due to facet joint degeneration and dislocation

Each vertebra has a pair of articular surfaces located on the left side and a pair of articular surfaces located on the right side. Each pair includes a superior articular surface that forms a facet joint with the adjacent higher vertebra and an inferior articular surface that forms a facet joint with the adjacent lower vertebra. Together the superior and inferior articular surfaces of adjacent vertebrae form a facet joint. Facet joints are synovial joints that are surrounded by a capsule of connective tissue with synovial fluid nourishing and lubricating the joint. The facet joint is formed by the union of two facets, one from each of an upper and lower vertebra. Each vertebra in the spine has two facets, one on the left and one on the right. Consequently, at the union of each coupling of vertebra there are two facet joints. The facet joints are located at the back (posterior) of the spine. The joint surfaces are coated with cartilage and are surrounded by a connective tissue, and each joint produces fluid to nourish and lubricate the joint.

Facet screw fixation is when the facet joint is significantly immobilized. The joint surfaces are coated with cartilage allowing the joints to articulate relative to one another. With adjacent intervertebral bodies fused together in a spinal fusion procedure, the fixation of the facet provides an additional supplement to spinal fusion. Facet fixation can also be employed as a stand-alone procedure or primary means to stabilize the spine without fusion of intervertebral bodies. Facet fixation can also serve to augment pedicle screw fixation or other posterior instrumentation on one side of the spine.

Typically, in facet fixation, a single facet screw is inserted directly across the facet joint to fix or limit the motion of the facet joint. In one variation of facet screw fixation called translaminar facet screw fixation, screws are inserted from the base of the spinous process on the contralateral side and through the lamina to traverse the facet joint indirectly and into the pedicle of the successively inferior vertebra. In translaminar facet screw fixation, the translaminar facet screws are longer than the screws used in direct facet fixation. To fixate both facet joints of a motion segment, two translaminar facet screws are placed in crisscross fashion across the lamina. Other procedures involve positioning an implant in the facet joint between the articular faces which may require removal of bone and the implantation of bone graft and/or growth material with or without a facet screw across the joint. Where the lamina is weak, translaminar facet screws can toggle and even break the lamina and destabilize the joint. However, facet screw fixation offers significant advantages when compared to pedicle screw fixation. For example, bilateral facet fixation uses only two screws per level replacing four screws, two rods and associated caps used in pedicle screw fixation. Facet fixation requires less hardware, less operating time and is easier to implant.

Furthermore, facet screw strength and stability is comparable to traditional pedicle screws. Facet screws also offer the potential reduction in fluoroscopic imaging resulting in less exposure to radiation due to less hardware to implant. Furthermore, facet fixation preserves adjacent level anatomy compared to pedicle screw fixation. Because of these and other advantages offered by facet screws, they are now being implanted on a regular basis. In order to further improve upon the use of such facet screws, an improved facet screw and a minimally invasive method for accurately and repeatedly placing facet screws across the facet joints is needed. This invention provides an improved facet screw and novel method of implantation.

What is needed are improved spinal stabilization, devices and methods, capable of providing facet joint support while allowing near normal motion of those facet joints on any spinal section.

### SUMMARY OF THE INVENTION

The present invention addresses the need to elevate facet related pain caused by the facet faces being near bone on bone and or misaligned. By providing an improved facet screw apparatus, and methods for the delivering that screw, and securing the vertebral facets while allowing the center of the screw to become flexible, thereby allowing the facets to move more normal. The improved device and methods of the present invention specifically address facet joint related pain but may have other significant applications not specifically mentioned herein. For purposes of illustration only, and without limitation, the present invention is discussed in detail with reference to the treatment of damaged facet joints of the human spinal column.

The present invention comprises a proximal mesh engagement mechanism, an adhesion ring, a distal mesh engagement mechanism, a drive socket bonding core, a flexible mesh, a stabilizing plug and a flexible separation donut. A specially designed drill mechanism is used to pre-drill the holes in the facets for installing the present invention novel facet screw. A solid tightening wrench that is utilized clinically to initially install the novel facet screw into two corresponding spinal facets.

The present invention aims at addressing the clinical condition of the patient while allowing the patient to maintain mobility not common with fusion procedures. The device and procedure allow the restoration of the relative spacing between the spinal facets, within the facet joint, alleviating the bone on bone contact that is common in degenerative facet joints and often the source of pain, while allowing relative motion between the facets to continue post-operatively. The proper alignment and spacing of the facet joint may improve other spine related pain.

While there are other inventions in existence that attempt to address facet degeneration and restore stability thru the use of screws and bone fusion, the subject device offers the benefit of requiring no bony resection and or fusion. This advantage provides the opportunity for the patient to rely more on those anatomical structures unaffected by degeneration while providing for very little morbidity in the surgical procedure. The less invasive procedure of installing a screw and spacer will undoubtedly benefit the patient's recovery time and should be less costly.

### BRIEF DESCRIPTION OF THE DRAWINGS

FIG. 1 is a perspective view of a spinal vertebral (10) showing the presents of the novel flexible facet screw apparatus inserted between two adjoining facet facets with a cushioning spacer between the facet faces.

FIG. 2 is a perspective exploded view of the assembled main components of the novel flexible facet screw apparatus, and cushioning spacer, without the screw's spine/drive wrench and the stabilizing flexible core plug.

FIG. 3 is a exploded perspective view of the main assembled components of the novel flexible facet screw apparatus showing the proximal facet engagement mechanism, an adhesion ring, the flexible tubular shaped mesh, the distal engagement mechanism, and inner lumen proximal mesh engagement mechanism with drive socket.

FIG. 4 is a side view taken of the specialize step drill that can be included in the kit.

FIG. 5 is an exploded perspective view of the main assembled components of the novel flexible facet screw apparatus showing the proximal facet engagement mechanism, an adhesion ring, the flexible tubular shaped mesh, the distal engagement mechanism, and internal distal bonding screw/core with drive socket cavity, a drive socket set pin, cushioning spacer apparatus, the screw's metal spine/drive wrench and flexible stabilizing core plug.

FIG. 6 is a side perspective cross-sectional view of the novel flexible facet screw apparatus delivered into position with the cushioning spacer apparatus sandwiched between two facet facets.

FIG. 7 is a flow chart showing the clinical steps necessary delivering and inserting the novel flexible facet screw apparatus.

### DESCRIPTION OF THE EMBODIMENTS

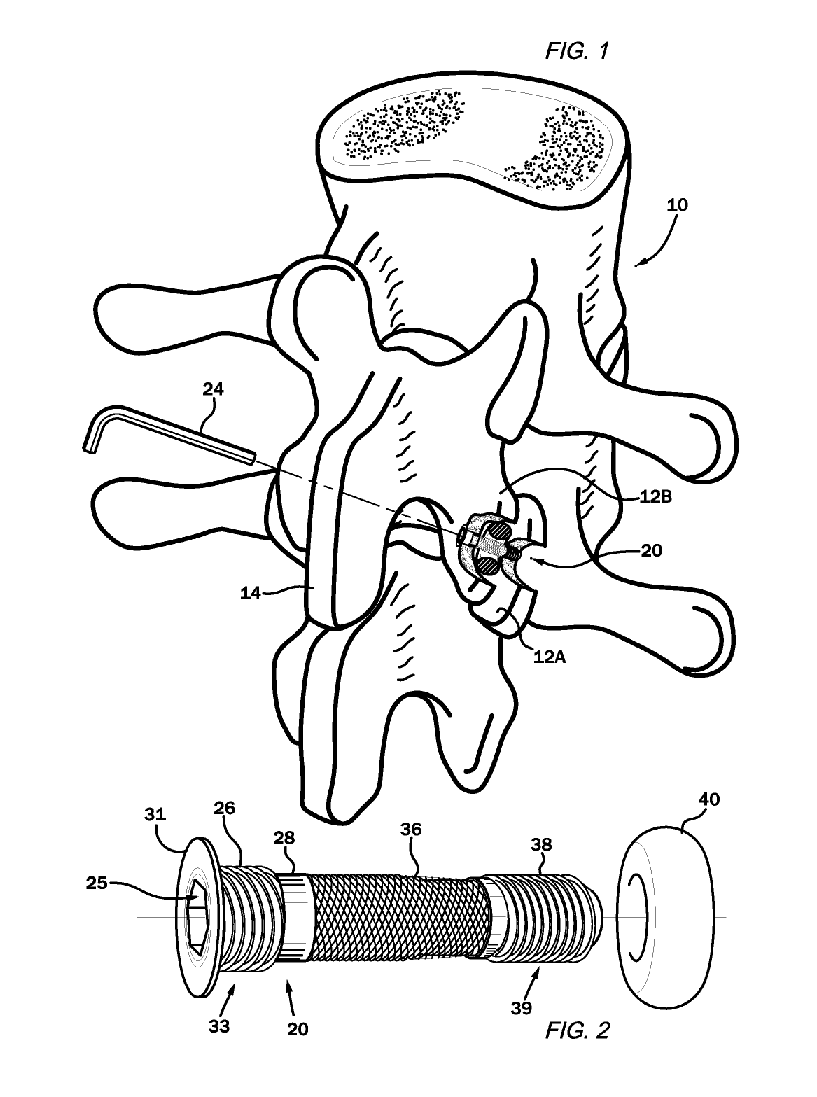

Now referring to FIG. 1, the flexible facet screw is a perspective view of a spinal vertebral 10 showing the presents of the novel flexible facet screw 20 apparatus inserted between two adjoining facet facets 12a, 12b with a cushioning spacer between the facet faces. FIG. 1 also show present invention tightening wrench 24 that is used to assemble the present invention 20 once placed into the pre-drilled facets.

FIG. 2 is a perspective side view of the assembled main components of the novel flexible facet screw apparatus 20, and cushioning spacer 40. The flexible facet screw 20 is comprised of multiple components that functions initially like a standard solid facet screw during delivery but once located properly the center section becomes a flexible facet screw apparatus 20 due to its central flexible components. The flexible facet screw apparatus 20 comprises a proximal mesh engagement mechanism 26 having a plurality of screw treads 33 and a shoulder ridge 31. An adhesion or bonding surface for which adhesion ring 28 that is designed to capture the distal end of the flexible tubular shaped mesh apparatus 36 between the bonding surface and the inside surface of the adhesive ring 28. The flexible tubular shaped mesh apparatus 36 is then bonded by adhesive, crimp or press fit or similar technology such the engagement between the proximal adhesion ring 28 and to the bonding surface of the proximal mesh engagement mechanism 26 is relatively permanent.

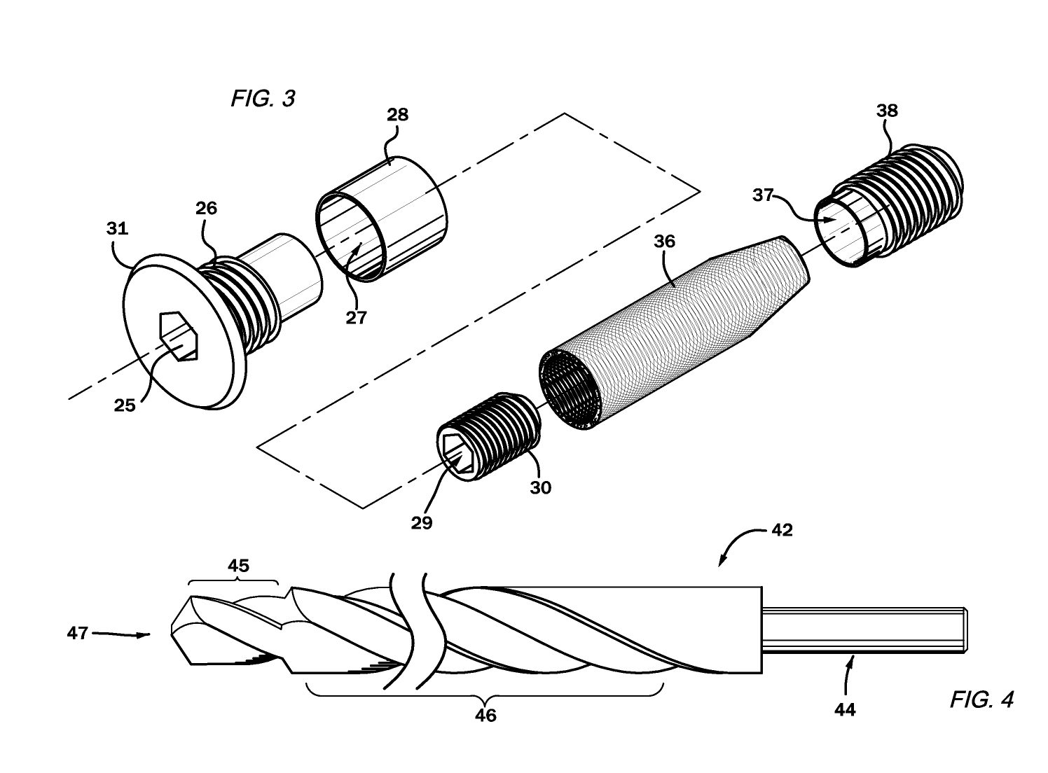

A distal bonding screw/core 30 with drive socket cavity 29 (shown in FIG. 3) is designed to engage, by press fit or internal screw threads means, the inside of the flexible tubular shaped mesh apparatus 36 into the inside surface 37 of the distal facet engagement mechanism 38, thereby bonding the flexible tubular shaped mesh apparatus to the distal end of the screw and positioning the drive socket 30 into the distal end of the screw. The initial solid phase of the novel flexible facet screw apparatus 20 occurs when the removable wrench 24 is inserted through the proximal mesh engagement mechanism 25 cavity 25, treaded through the flexible mesh and inserted in to the drive socket cavity 29 of the distal bonding screw/core 30. After located properly between two facets, the solid wrench is removed rendering the central area of the novel flexible facet screw apparatus 20 pliable and flexible.

Now referring to FIG. 3, shown are most of the main components of the novel flexible facet screw apparatus 20. The proximal mesh engagement mechanism 26 has a plurality of screw treads 33 and a shoulder ridge 31. The proximal adhesive ring 28 then secures the proximal end of the flexible mesh 36 to the proximal mesh engagement mechanism 26. An internal distal bonding screw/core 30 with drive socket cavity 29 is shown with threads to aid in securing the distal end of the flexible mesh but it is contemplated by the Applicant the secure means could be adhesive, crimp, press fit or other technology. The distal mesh engagement mechanism 38 has a plurality of threads 39 and in internal lumen 37 that receives the distal end of the flexible mesh 36 and distal bonding screw core 30.

FIG. 4 is a side view taken of the specialize step drill 42 that can be included in the kit. The drill bit 42 has a shank 44 that is designed to be tightened within the collet of an appropriate drill apparatus. The drill bit 42 is specifically designed to provide access for the present novel flexible facet screw apparatus 20 to be inserted between two adjacent facets. The drill bit 42 has a fixed larger size diameter section 46, a sharp tip 47, and a reduced diameter section 45. The drill bit 42 is appropriately sized to allow proximal mesh engagement mechanism 26 with plurality of screw threads to 33 engage the first vertebral facet and the distal mesh engagement mechanism 38 plurality of screw threads 39 to engage the second vertebral facet. The distal end of drill bit has a reduced diameter size such that it fits through the lumen created in the first drill facet. Thus the proximal mesh engagement mechanism 26 with plurality of screw threads 33 is slightly larger than the distal mesh engagement mechanism 38 with screw threads 39 which allows for facilitating delivery of the novel flexible facet screw apparatus 20. The engagement of the plurality of threads is accomplished by the rotation of the metal spine/drive wrench 24. The metal spine/drive wrench 24 is rotated until the shoulder 29 of the proximal mesh engagement mechanism 26 engages the outside surface of the first facet.

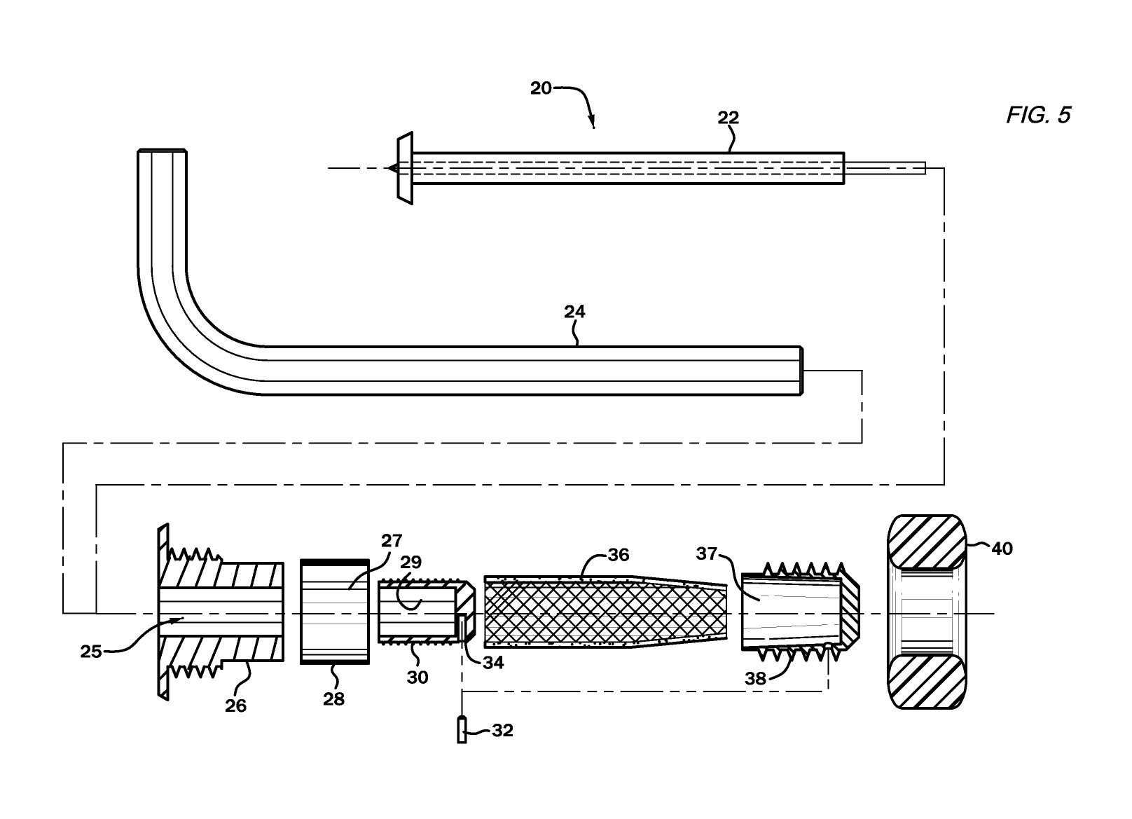

Now referring to FIG. 5, which is an exploded perspective view of the main assembled components of the novel flexible facet screw apparatus 20 showing, as discussed in detail under FIGS. 2 and 3, the proximal facet engagement mechanism 26, an adhesion ring 28, the flexible tubular shaped mesh 36 the distal engagement mechanism 38, and internal distal bonding screw/core 30 with drive socket cavity 29, a drive socket set pin 32, cushioning spacer apparatus 40, the screw's metal spine/drive wrench 24 and flexible stabilizing core plug 22.

Further shown in FIG. 5 is a set pin 32 is installed into the hole in the distal screw end, into a hole 34 of the internal distal bonding screw/core 30 and within the hole 35 of the distal facet engagement mechanism 38 to secure the distal end of the flexible tubular shaped mesh apparatus 36 securely into place. The distal facet engagement mechanism 38 includes a plurality of screw threads 39 that are designed to engage corresponding lumen that is drilled into the facet. The flexible facet screws contain a metal spine/drive wrench cavity 25 that corresponds to a removable wrench 24 that is made titanium, stainless steel, or other surgical metal.

The screw's metal spine/drive wrench 24 allows the screw to act as a solid structure during installation and until the distal core/screw 30 is fully engaged within the distal facet, the metal spine/drive wrench 24 is removed rendering the center area of the novel flexible facet screw apparatus 20. After properly localized, a stabilizing flexible core plug 22 is then inserted into the inner lumen through the proximal facet engagement mechanism 26 and inner lumen 25, through the inner lumen of the flexible tubular shaped mesh 36, through the internal distal bonding screw/core 30 with drive socket cavity 2 and then becomes engaged in the inner lumen 37 and secured to the distal facet engagement mechanism 38. To facilitate removal and add stability the stabilizing flexible core plug 22 compromises a Kevlar or other high strength material or fiber to improve the strength needed for clinical applications.

When the metal spine/drive wrench 24 is used to rotate the both the proximal and distal facet engagement mechanisms 26, 38 and when this operation is completed, is removed and replaced by the stabilizing flexible core plug 22 which functions with the flexible tubular mesh section 36 to becomes flexible with stabilization and structural integrity. The flexible section should be aligned between the facet surfaces which contains the cushioning spacer 40 that fits between the two facets.

The proximal and distal facet engagement mechanism 26, 38 adhesion bonding ring 28, internal distal bonding screw/core 30 and metal spine/drive wrench 24 may be fabricated from stainless steel, titanium metals, or engineering plastics with structural integrity generally thermo-set polymers or ABS, HDPE, PET, PE, PVC, HIPS, polyamides, polycarbonates, and combination thereof.

The substantially tubular shaped flexible mesh 36 may be formed of a woven, knitted, or braided material and may be made of carbon fiber, PEEK (polyetheretherketone), Nylon, Dacron, synthetic polyamide, polypropylene, expanded polytetrafluroethylene (e-PTFE), polyethylene and ultra-high molecular weight fibers of polyethylene (UHMWPE) commercially available as Spectra™ or Dyneema™, as well as other high tensile strength materials such as Vectran™, Kevlar™, natural or artificially produced silk and commercially available suture materials used in a variety of surgical procedures. Alternatively the flexible tubular mesh may be formed from metallic materials, for example, stainless steel, elgiloy, Nitinol, or other biocompatible metals or composites of non-metallic and metallic materials.

An elastomeric facet joint spacer 40 specific for each unique facet joint, is designed to coaxially traverse the outer surface of the flexible tubular shaped mesh and provide a cushioning effect between the two facet facets. The elastomeric facet joint spacer apparatus 40 can be fabricated from a polymer with viscoelasticity generally having a low Young's modulus and high failure strain and can be formed from natural or synthetic polyisoprene, polybutadiene, chloroprene, butyl rubber, nitrile rubber, ethylene propylene rubber, polyacrylic rubber, silicone rubber, hydrogels, and urethane materials.

The stabilizing flexible core plug 22 can be fabricated from a polymer with viscoelasticity generally having a low Young's modulus and high failure strain and can be formed from natural or synthetic polyisoprene, polybutadiene, chloroprene, butyl rubber, nitrile rubber, ethylene propylene rubber, polyacrylic rubber, silicone rubber, hydrogels, and urethane materials.

As a kit, the flexible facet screw device also can include a specially designed step drill and various sizes cushioning spacers.

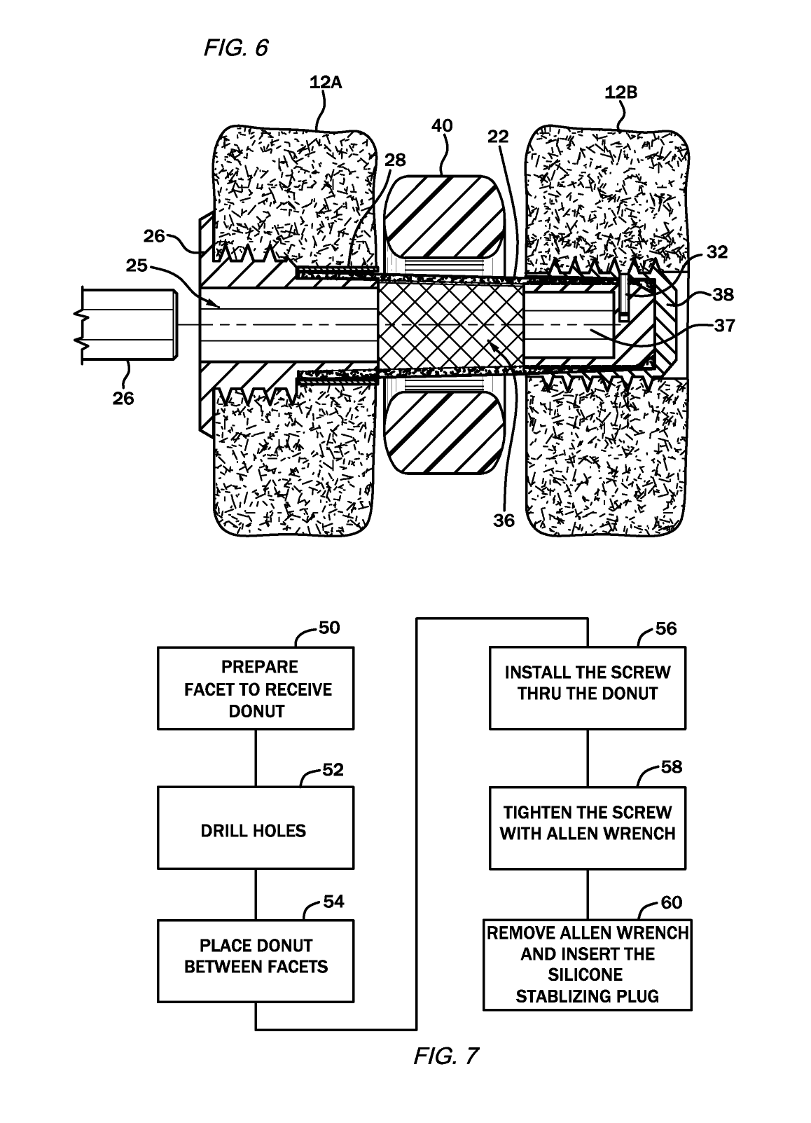

FIG. 6 is a side perspective cross-sectional view of the novel flexible facet screw apparatus 20 delivered into position with the elastomeric facet joint spacer 40 sandwiched to provide cushioning between two facet facets 12a, 12b, after the metal spine/drive wrench 24 has been removed and before the flexible stabilizing core plug is inserted.

As shown in this FIG. 6, the proximal facet engagement mechanism 26 is threaded within a drilled lumen in facet 12a with the shoulder 31 engaging the outside surface of the facet 12a. The proximal end of the flexible tubular shaped mesh apparatus 36 is sandwiched and engaged between the outside surface of the proximal facet engagement mechanism 25 and the inside surface of the adhesion bonding ring 28. Also shown is a lumen 25 within the proximal facet engagement mechanism 26.

Shown between facet 12a and 12b, is the substantially tubular shaped flexible mesh 36 in coaxial cooperation with the elastomeric facet joint spacer 40.

The distal facet engagement mechanism 38 is threaded within a drilled lumen of facet 12b. The Internal distal bonding screw/core 30, is further help in place with set pin 32.

Also shown on FIG. 6, is the stabilization flexible core plug 22 located outside the proximal facet engagement mechanism 26 and in position to be inserted through the proximal facet engagement mechanism 26 lumen 25, lumen through substantially tubular shaped flexible mesh 36, and lodged into lumen 37 of the distal facet engagement mechanism 38.

FIG. 7 is a flow chart showing the clinical steps necessary delivering and inserting the novel flexible facet screw apparatus. In the first step 50, the facet joint is prepared to be drilled and receive the novel flexible facet screw apparatus Step 52 defines the step drilling, using the specifically designed drill bit, the different diameter sized holes in the two adjacent facet for providing stabilization between the two facets. After the holes are dilled in the facets, step 54 defines the process of inserting the elastomeric facet joint spacer 40 between the two drilled facets and aligned with holes. Step 56 defines the process of using the spine drive wrench 24 to insert the distal facet engagement mechanism 38 with attached substantially tubular shaped flexible mesh 36 and proximal facet engagement mechanism 26 through the first facet hole, coaxially through elastomeric facet joint spacer 40, and then into the second drilled hole in the second facet. Step 58 defines rotating the spine drive wrench 24, that has been insert into the distal facet engagement mechanism cavity 37 and/or inserted into the lumen 25 of the proximal facet engagement mechanism 26 to securing engage these mechanisms 26, 38 into the facets and securing the elastomeric facet joint spacer into place. Step 60 defined the process of then removing the spine drive wrench 24 and install the stabilizing flexible core plug 22.

The present invention, method of use, and variations of its embodiments is summarized herein. Additional details of the present invention and embodiments of the present invention may be found in the Detailed Description of the Preferred Embodiments and Claims below. These and other features, aspects and advantages of the present invention will become better understood with reference to the following descriptions and claims.

1. An novel facet screw apparatus comprising;
a proximal mesh engagement mechanism;
an adhesion ring;
a distal mesh engagement mechanism;
a drive socket bonding core;
a flexible mesh;
a flexible stabilizing plug; and
a flexible separation donut.

2. A novel facet screw apparatus as recited in claim 1, whereby said facet screw apparatus is designed to be initially used and delivered as a solid type facet screw, but after installation, becomes flexible when a solid core drive wrench is exchanged for said flexible stabilizing plug.

3. A novel facet screw apparatus as recited in claim 1, whereby said facet screw apparatus is designed to stabilize the spinal facet joints while allowing normal facet joint mobility.

4. A novel facet screw apparatus as recited in claim 2, whereby said facet screw apparatus is designed to retain a cushioning spacer between the spinal facet joints while maintain the normal facet spacing and position and retaining facet joint mobility.

5. An novel facet screw kit comprising;
a novel facet screw having a proximal mesh engagement mechanism, an adhesion ring, a distal mesh engagement mechanism, a drive socket bonding core, a flexible mesh, a flexible stabilizing plug; and a flexible separation donut;
a specially designed drill mechanism is used to pre-drill the holes in the facets for installing the novel facet screw;
a solid tightening wrench that is utilized clinically to initially install the novel facet screw into two corresponding spinal facets.

6. A novel facet screw kit as recited in claim 5, whereby said facet screw apparatus is designed to be initially used and delivered as a solid pedicle type facet screw, but after installation becomes flexible when a solid core drive wrench is exchanged for said flexible stabilizing plug.

7. A novel facet screw kit as recited in claim 5, whereby said facet screw apparatus is designed to stabilize the spinal facet joints while allowing normal facet joint mobility.

8. A novel facet screw kit as recited in claim 5 whereby said facet screw apparatus is designed to retain a cushioning spacer between the spinal facet joints while maintain the normal facet spacing and position and retaining facet joint mobility.

9. A method of clinically using novel facet screw kit to adjoin a pair of facets comprising;
preparing a pair of facets to receive a flexible separation donut;
drilling a hole through the two adjoining facets, said drilling hole creating a series of threads within the drilled hole;
placing a flexible separation donut between the drilled facets;
installing the novel facet screw having a proximal mesh engagement mechanism, an adhesion ring, a distal mesh engagement mechanism, a drive socket bonding core, a flexible mesh, a flexible stabilizing plug;
tightening the novel facet screw with the tightening wrench; and
removing the tightening wrench and replacing with a flexible stabilization plug.

10. A method of clinically using novel facet screw kit to adjoin a pair of facets as recited in claim 9, whereby said facet screw apparatus is designed to be initially used and delivered as a solid type facet screw, but after installation becomes flexible when a solid core drive wrench is exchanged for said flexible stabilizing plug.

11. A method of clinically using novel facet screw kit to adjoin a pair of facets as recited in claim 9, whereby said facet screw apparatus is designed to stabilize the spinal facet joints while allowing normal facet joint mobility.

12. A method of clinically using novel facet screw kit to adjoin a pair of facets as recited in claim 9, whereby said facet screw apparatus is designed to retain a cushioning spacer between the spinal facet joints while maintain the normal facet spacing and position and retaining facet joint mobility.
